(19) United States
(12) Patent Application Publication
(10) Pub. No.: US 2020/0139340 A1
(43) Pub. Date: May 7, 2020

(54) DEVICE FOR GENERATING ULTRA PURE 1-METHYLCYCLOPROPENE

(71) Applicant: Janssen Pharmaceutica NV, Beerse (BE)

(72) Inventors: Wendy Condy Ria SCHOTSMANS, Beerse (BE); Vincent Jan VANTOL, Eindhoven (NL); Frank BOON, Eindhoven (NL)

(21) Appl. No.: 16/625,043

(22) PCT Filed: Jun. 21, 2018
(86) PCT No.: PCT/EP2018/066510

§ 371 Date: Dec. 20, 2019

(30) Foreign Application Priority Data
Jun. 21, 2017 (EP) 17177030.8

(51) Int. Cl. B01J 19/24; C07C 1/32; A01N 27/00; A01N 3/00

(52) U.S. Cl. CPC B01J 19/245; C07C 1/321; C07C 13/04; A01N 3/00; A01N 27/00

(57) ABSTRACT

The present invention relates to a device for generating ultra pure 1-methyl-cyclopropene (1-MCP) by using an improved carrier gas flow control system. The invention also relates to the use of a 1-MCP generating device for inhibiting the action of ethylene which accelerates the ripening process of plants such as fruits, flowers, vegetables and the like. Furthermore the invention encompasses a method for treating and storing harvested agricultural products using said 1-MCP generating device.

### TECHNICAL FIELD

The present invention relates to a device for generating ultra pure 1-methyl-cyclopropene (1-MCP) by using an improved carrier gas flow control system. The invention also relates to the use of a 1-MCP generating device for inhibiting the action of ethylene which accelerates the ripening process of plants such as fruits, flowers, vegetables and the like. Furthermore the invention encompasses a method for treating and storing harvested agricultural products using said 1-MCP generating device.

### BACKGROUND

Plant growth and fruit ripening are affected by several factors including plant hormones that regulate a wide variety of cellular processes. A well-known plant hormone is ethylene that mediates growth phenomena in plants through its interaction with specific ethylene receptors in plants. Many compounds other than ethylene interact with this receptor: some mimic the action of ethylene, others prevent ethylene from binding and thereby counteract its action.

Cyclopropene derivatives such as 1-methylcyclopropene (1-MCP) can bind tightly to the ethylene receptors in plants, thereby blocking the effects of ethylene which results in maintaining the freshness of plants and flowers or the prevention of ripening of fruits.

1-MCP (1-methylcyclopropene) is a volatile gas at standard temperature and pressure which provides for easy treatment of agriculture products in storage space. It is however chemically unstable when not stored at a low temperature (below −100° C.) and can easily undergo loss of its chemical properties through dimerization etc.

In order to solve the problems associated with the storage of 1-MCP, various solutions have been provided. WO-00/10386 discloses the encapsulation of 1-MCP into cyclodextrines whereby the 1-MCP is liberated by heating the 1-MCP/cyclodextrine complex. Another solution is provided by WO-2007/058473 that discloses a device for the in-situ chemical generation of 1-MCP whereby the device stores the 1-MCP chemical precursor and activation reagent in two separate storage containers and through a chemical reaction the 1-MCP is synthesized.

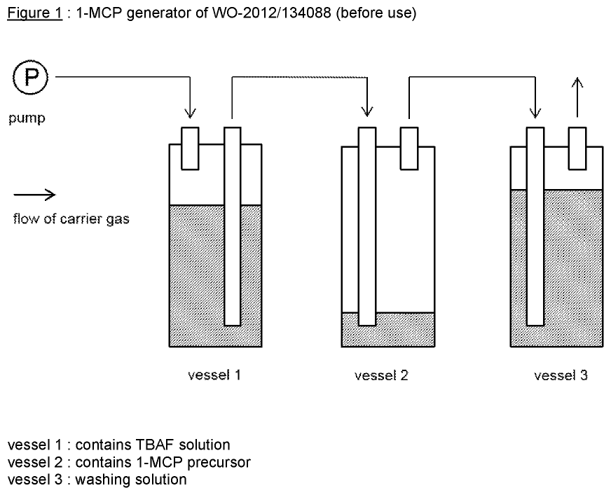

WO-2012/134088 discloses a device as depicted in FIG. 1 for generating 1-MCP which device comprises a first vessel containing tetrabutylammonium fluoride (TBAF) dissolved in DMF, a second vessel containing a solution of trans-1-methyl-1-(methanesulfonyloxy)-2-(butyldimethylsilyl)cyclopropane (i.e. the 1-MCP precursor), and a carrier gas that is introduced into the first vessel to transfer the TBAF containing solution into the second vessel containing the 1-MCP precursor solution whereby a chemical reaction yields 1-MCP that is moved by the carrier gas to a third vessel where it is cleaned before the carrier gas takes the 1-MCP to the outside. The carrier gas is air coming from an electric bubble generator for aquarium fish having a flow rate from 100 to 200 ml/min.

WO-2005/080267 describes a process and a reactor for the production of chloramine wherein the flow of the reagent gasses and carrier gas is controlled by mass flow controllers. WO-2016/053201 describes a method and system for producing olefins by dehydrogenating vaporized ethanol delivered by a nitrogen flow to a fixed bed reactor wherein the nitrogen flow is controlled by a mass flow controller. US-2006/0037644 describes a mass flow controller capable of supplying always stably at desired flow rate in spite of pressure fluctuations at either upstream side of downstream side of the mass flow controller.

### Technical Problem

The pump for generating the carrier gas in the 1-MCP generating device disclosed in WO-2012/134088 is an electric bubble generator for aquarium fish and suffers in the field from significant variations in flow rate which are likely related to changes in the ambient temperature, air pressure, humidity, and ageing of the rubber membrane in the membrane pump of the aquarium bubble generator.

Depending upon the size of the 1-MCP generating device and the amount of 1-MCP needed, the flow rate of the carrier gas needed can range from 20 ml/min up to 200 ml/min and such low flow rates are difficult to regulate reliably. When the flow rate of the carrier gas is too high for a given size of the 1-MCP generating device, there is an increasing possibility of discharging impurities, such as small amounts of solvent or reaction by-products, together with 1-MCP to the outside. When the flow rate of the carrier gas is too low, the 1-MCP generator needs to run longer to produce enough 1-MCP to treat the agriculture products in storage space with an increasing uncertainty whether sufficient 1-MCP has been released to treat all agriculture products.

A first attempt to obtain a reproducible and constant flow rate of the carrier gas with minimal deviation from the desired flow rate used a mechanical needle valve. However this set-up required a daily and cumbersome calibration of the needle valve to ensure the carrier gas had the desired flow rate.

Hence there is a need to have a 1-MCP generating device with a well-controlled, reproducible and constant flow rate of the carrier gas.

### Technical Solution

It has now been found that a well-controllable, reproducible and constant flow rate of carrier gas in a 1-MCP generating device can be obtained by measuring the flow rate of the carrier gas with a mass flow sensor and regulating the pump through an electronic flow control circuit. Furthermore it has also been found that the working of the mass flow sensor can be further improved by placing a flow restrictor between the pump and the mass flow sensor.

### DETAILED DESCRIPTION

A mass flow sensor, also known as an inertial flow meter, is a device that measures mass flow rate of a gas traveling through a tube. The mass flow rate is the mass of the gas traveling past a fixed point per unit time. The mass flow sensor does not measure the volume per unit time (e.g., cubic meters per second) passing through the device; it measures the mass per unit time (e.g., kilograms per second) flowing through the device. The output of a mass flow sensor, i.e. the flow rate signal, is usually expressed as SCCM (Standard Cubic Centimeters per Minute) a flow measurement term indicating cm³/min at a standard temperature and pressure (i.e. Tₙ=0° C., Pₙ=1.01 bar).

Commercially available mass flow sensors are e.g. the AWM3000 series supplied by Honeywell.

### SPECIFIC EMBODIMENTS

Description of the drawings:

FIG. 1: 1-MCP generator of WO-2012/134088 (before use) whereby vessel 1 contains TBAF solution and vessel 2 contains 1-MCP precursor solution

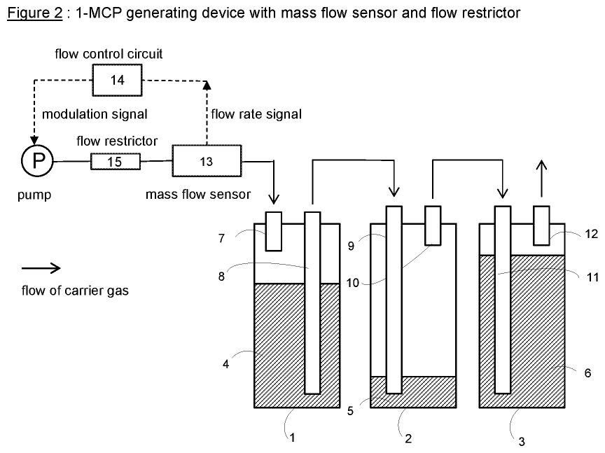

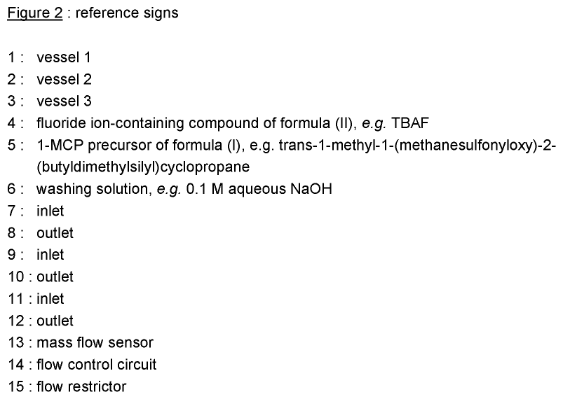

FIG. 2: 1-MCP generating device in accordance with the present invention wherein a mass flow sensor is used to regulate the flow rate of carrier gas produced by the pump and a flow restrictor is present between the pump and the mass flow sensor

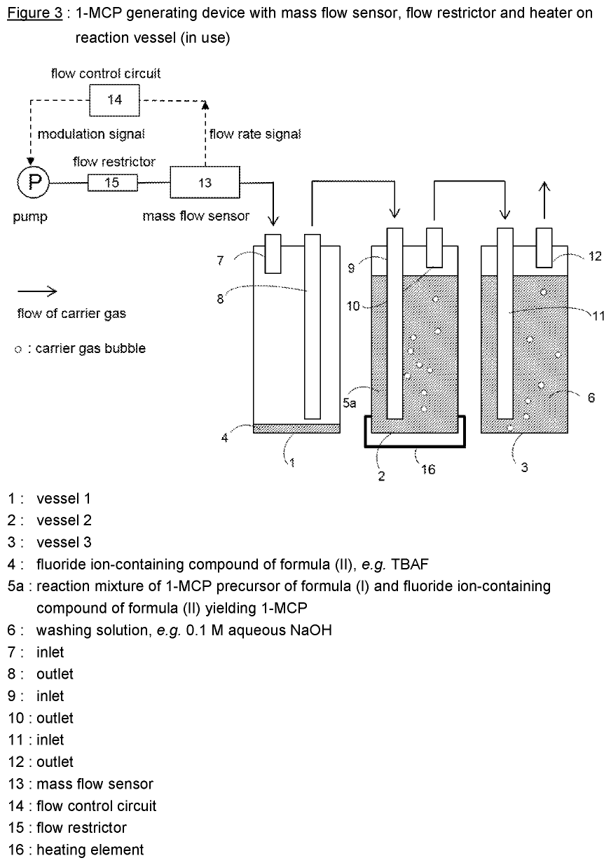

FIG. 3: 1-MCP generating device in accordance with the present invention further equipped with a heating element on the second vessel

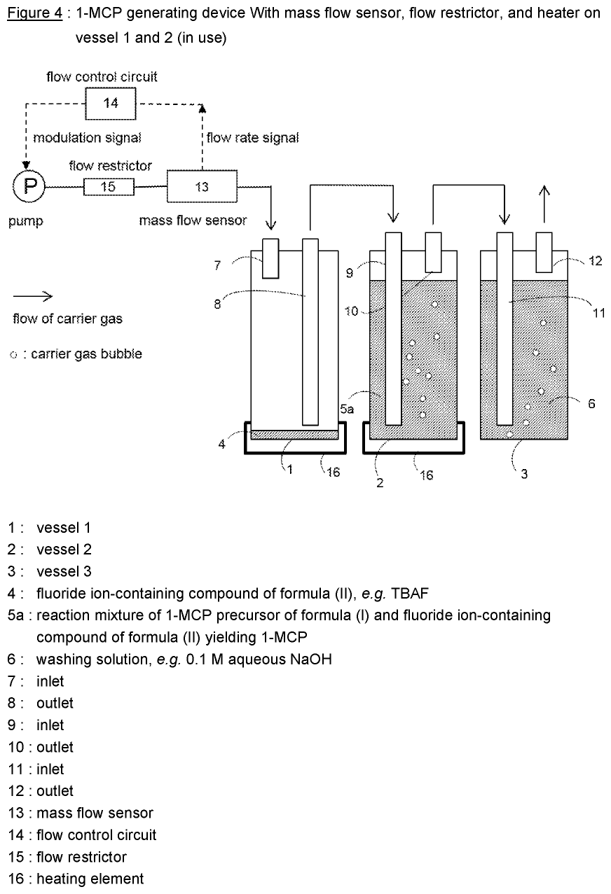

FIG. 4: 1-MCP generating device in accordance with the present invention further equipped with a heating element on vessel 1 and 2

FIGS. 2, 3, and 4 are drawings illustrating the design and the operation of a 1-MCP generating device according to an embodiment of the present invention. In the drawings, like reference numerals denote like elements of the 1-MCP generating device.

FIG. 2 is a drawing illustrating an embodiment of a 1-MCP generating device wherein the flow rate of the carrier gas does not deviate more than 20% from the desired flow rate, comprising:

a first vessel (1) comprising an inlet (7), an outlet (8), and (4) a fluoride ion-containing compound of formula (II),

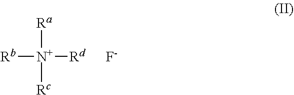

wherein R^{a}, R^{b}, R^{c}, R^{d} are each independently selected from C₁₋₂₀alkyl, phenyl and naphthyl;
a second vessel (2) comprising an inlet (9), an outlet (10), and (5) a 1-MCP precursor of formula (I)

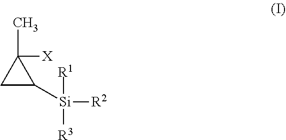

wherein X is halogen, or C₁₋₆alkylS(O)₂O—;
R¹, R², R³ are each independently selected from hydrogen, C₁₋₆alkyl, phenyl, C₁₋₆alkyloxy, and halogen;
a third vessel (3) comprising an inlet (11), an outlet (12), and a washing solution (6);
a pump (P) to supply a carrier gas that is introduced into the first vessel (1) to transfer the (4) fluoride ion-containing compound of formula (II) to the second vessel (2) where said (4) fluoride ion-containing compound of formula (II) reacts with the (5) 1-MCP precursor of formula (I) and the resulting 1-MCP is transferred with the carrier gas to the third vessel (3) where it bubbles through the washing solution (6) before the carrier gas with the 1-MCP is released to the outside;
characterized in that a mass flow sensor (13) is placed between the pump (P) and the first vessel (1) to regulate the flow rate of the carrier gas and a flow restrictor (15) is placed between the pump (P) and the mass flow sensor (13).

The mass flow sensor (13) measures the flow rate of the carrier gas and provides a flow rate signal to the flow control circuit (14) that provides the pump (P) with a modulation signal to regulate the flow rate of the carrier gas so that it does not deviate more than 20% from the desired flow rate.

It has been found that the pump (P), especially when a membrane pump is used, can generate a sound wave in the carrier gas that interferes with the correct working of the mass flow sensor (13). This problem can be solved by including a flow restrictor (15) between the pump (P) and the mass flow sensor (13). Such a flow restrictor (15) can be a short piece of narrow bore tubing. For instance the diameter of the narrow bore tubing used as flow restrictor is 1/10 of the diameter of the tubing used to connect the pump (P) to the mass flow sensor (13). The flow restrictor (15) can also be an orifice disc with a small hole wherein said hole has a diameter ranging from 0.10 mm to 0.25 mm, in particular 0.15 mm.

FIG. 3 is a drawing illustrating a further embodiment of a 1-MCP generating device comprising in addition to the device of FIG. 3 a heating element (16) to heat the second vessel (2) during the reaction between the 1-MCP precursor (5) and the fluoride-ion containing compound (4). When the 1-MCP generating device is used in a cooled storage facility, the second vessel (2) should be heated to facilitate reaction between the 1-MCP precursor (5) and the fluoride-ion containing compound (4). Also performing the 1-MCP generating reaction at an elevated temperature that is independent of the ambient temperature provides for a more reproducible production of 1-MCP each time the 1-MCP generating device is used. In particular the second vessel (2) is heated to a temperature ranging from 30° C. to 50° C., more particularly the temperature should range from 40° C. to 45° C. Furthermore FIG. 4 is a drawing illustrating an embodiment of a 1-MCP generating device in operation whereby the fluoride-ion containing compound (4) and 1-MCP precursor (5) are mixed in the second vessel (2).

FIG. 4 is a drawing illustrating a further embodiment of a 1-MCP generating device comprising in addition to the device of FIG. 4 a heating element (16) to heat the vessel (1) prior to the reaction between the 1-MCP precursor (5) and the fluoride-ion containing compound (4).

The first vessel (1), the second vessel (2), and the third vessel (3) include respectively inlets (7), (9) and (11) and respectively outlets (8), (10) and (12). The first vessel (1), the second vessel (2), and the third vessel (3) are connected to each other through a tube via the respective inlets and outlets. When the 1-MCP generating device is in operation, carrier gas is supplied through a tube to the inside of the first vessel (1) through the inlet (7) of the first vessel (1), and the outlet (8) of the first vessel (1) is connected to the inlet (9) of the second vessel (2) through a tube. The outlet (10) of the second vessel (2) is connected to the inlet (11) of the third vessel (3) through a tube whereby the carrier gas transfers the 1-MCP to the outside via the outlet (12) of the third vessel (3).

In the 1-MCP generating device described in FIGS. 2 to 4 the first vessel (1) is used to store the fluoride ion-containing compound of formula (II) which is then transferred by the carrier gas to the second vessel (2) to mix with the 1-MCP precursor of formula (I) to prepare 1-MCP. 1-MCP can also be prepared in said device when the first vessel (1) contains the 1-MCP precursor of formula (I) and the second vessel (2) contains the fluoride ion-containing compound of formula (II). In practice the carrier gas does not transfer all of the content of the first vessel (1) to the second vessel (2) and a small amount is often left in the first vessel (1). Since the fluoride ion-containing compound of formula (II) is present in excess to the 1-MCP precursor of formula (I) it has no influence on the yield of 1-MCP if the content of vessel (1) is not completely transferred to vessel (2).

### GENERAL EMBODIMENTS

The present invention relates to a 1-methylcyclopropene (1-MCP) generating device wherein the flow rate of the carrier gas does not deviate more than 20% from the desired flow rate, comprising:

a first vessel comprising an inlet, an outlet, and a fluoride ion-containing compound of formula (II)

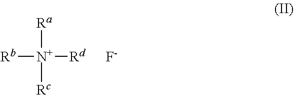

wherein R^{a}, R^{b}, R^{c}, R^{d} are each independently selected from C₁₋₂₀alkyl, phenyl and naphthyl;
a second vessel comprising an inlet, an outlet, and a 1-MCP precursor of formula (I)

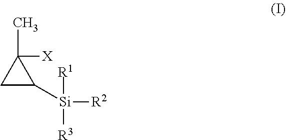

wherein X is halogen, or C₁₋₆alkylS(O)₂O—;
R¹, R², R³ are each independently selected from hydrogen, C₁₋₆alkyl, phenyl, C₁₋₆alkyloxy, and halogen;
a third vessel comprising an inlet, an outlet, and a washing solution;
a pump to supply a carrier gas that is introduced into the first vessel to transfer the fluoride ion-containing compound of formula (II) to the second vessel where said fluoride ion-containing compound of formula (II) reacts with the 1-MCP precursor of formula (I) and the resulting 1-MCP is transferred with the carrier gas to the third vessel where it bubbles through the washing solution before the carrier gas with the 1-MCP is released to the outside;
characterized in that the flow rate of the carrier gas is regulated by a mass flow sensor that provides a flow rate signal to a flow control circuit that provides the pump with a modulation signal to regulate the flow rate of the carrier gas and that a flow restrictor is present between the pump and the mass flow sensor.

The pump to supply the carrier gas can be a diaphragm or membrane air pump, a piston pump, a rotary vane pump, or any other device suitable for moving gases.

The mass flow sensor that measures the flow rate of the carrier gas provides a flow rate signal to the flow control circuit that provides the pump with a modulation signal to regulate the flow rate of the carrier gas so that it does not deviate more than 20% from the desired flow rate. A mass flow sensor suitable for use in the 1-MCP generating device of the present invention is e.g. the AWM3000 series supplied by Honeywell.

It has also been found that the flow of the carrier gas can be made more constant by introducing a flow restrictor in between the pump and the mass flow sensor. Due to the intermittent on/off working of the pump the carrier gas can have a fluctuating flow that can hamper the working of the mass flow sensor and flow control circuit to keep the desired flow rate within a 20% deviation. These fluctuations of the carrier gas flow can be greatly reduced by introducing a flow restrictor between the pump and the mass flow sensor. Such a flow restrictor can be a short piece of narrow bore tubing. For instance the diameter of the narrow bore tubing used as flow restrictor is 1/10 of the diameter of the tubing used to connect the pump to the mass flow sensor. The flow restrictor can also be an orifice disc with a small hole wherein said hole has a diameter ranging from 0.10 mm to 0.25 mm, in particular 0.15 mm.

In an embodiment the mass flow sensor is placed between the pump and the first vessel. The first vessel, the second vessel, and the third vessel include inlets and outlets that are connected through tubing in the desired sequence.

The 1-methylcyclopropene (1-MCP) is prepared by reacting a 1-MCP precursor of formula (I) with a fluoride ion-containing compound of formula (II) as depicted in the following reaction scheme:

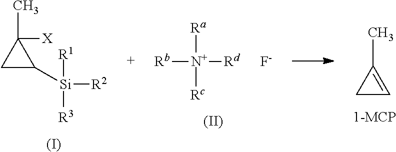

wherein X is halogen, or C₁₋₆alkylS(O)₂O—;
R¹, R², R³ are each independently selected from hydrogen, C₁₋₆alkyl, phenyl, C₁₋₆alkyloxy, and halogen;
R^{a}, R^{b}, R^{c}, R^{d} are each independently selected from C₁₋₂₀alkyl, phenyl and naphthyl.

As used in the foregoing definitions:
halogen is generic to fluoro, chloro, bromo and iodo;
C₁₋₆alkyl defines straight and branched chain saturated hydrocarbon radicals having from 1 to 6 carbon atoms such as, for example, methyl, ethyl, propyl, butyl, 1-methyl-ethyl, 2-methylpropyl, 2-methylbutyl, pentyl, hexyl and the like;
C₁₋₂₀alkyl is meant to include C₁₋₆alkyl and the higher homologues thereof having from 7 to 20 carbon atoms, such as, for example, heptyl, octyl, nonyl, decyl and the like.

In a preferred embodiment X is CH₃—SO₂—O—; R¹ and R² are methyl and R³ is n-butyl; and R^{a}, R^{b}, R^{c} and R^{d} are each n-butyl; whereby 1-MCP is prepared as follows:

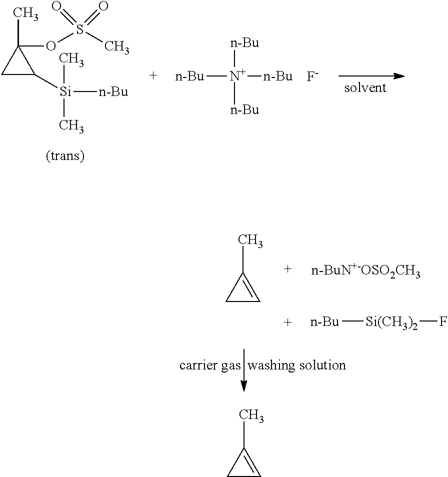

In another embodiment of the 1-MCP generating device of the present invention the second vessel is equipped with a heating element. When the 1-MCP generating device is used the second vessel is heated to a temperature ranging from 30° C. to 50° C., more particularly to a temperature ranging from 40° C. to 45° C.

The 1-MCP precursor of formula (I) may be used in a dissolved form in a solvent such as DMF, DMSO, or dimethylacetamide, or also used alone (i.e. not dissolved). A particular 1-MCP precursor of formula (I) is trans-1-methyl-1-(methanesulfonyloxy)-2-(butyldimethylsilyl)cyclopropane as used in the reaction scheme above.

The fluoride ion-containing compound of formula (II) may be used in a dissolved form in a solvent such as DMF, DMSO, or dimethylacetamide, rather than used alone. A preferred solvent is DMSO. The solvent may be used in an amount of from 0.5 to 3.0 times the amount of the fluoride ion-containing compound, but if only a small amount of 1-MCP is needed, the solvent may be used in an amount of 10 times the amount of the fluoride ion-containing compound. A preferred fluoride ion-containing compound of formula (II) is tetrabutylammonium fluoride (TBAF).

The 1-MCP precursor of formula (I) and the fluoride ion-containing compound of formula (II) may be simply mixed or only contacted with each other, thereby obtaining 1-MCP. To facilitate mixing, vessel (2) may be equipped with a mixing device.

The amount of the fluoride ion containing compound is 1 to 3 mol to 1 mol of the 1-MCP precursor. A preferred amount of fluoride ion containing compound is 2.7 mol to 1 mol of 1-MCP precursor.

The carrier gas used in the 1-MCP generating device can be air or any inert gas such as nitrogen.

Before the carrier gas releases the 1-MCP gas prepared by the reaction of the 1-MCP precursor of formula (I) and the fluoride ion-containing compound of formula (II) to the outside, the 1-MCP gas is passed through a washing solution that removes reaction byproducts such as halosilane or acidic byproducts such as HF by decomposition or neutralization. The washing solution is a basic aqueous solution prepared by dissolving NaOH, KOH, Na₂CO₃, NaHCO₃, K₂CO₃, KHCO₃, Na₂SiO₂, K₂SiO₂, sodium methanolate, sodium ethanolate, or sodium isopropanolate. A preferred washing solution is 0.1 M aqueous sodium hydroxide solution.

In general, 1-MCP has a sufficient effect in air even at a low concentration of 1 ppm or less, and thus, approximately 0.01 to 5.0 litre (0.45 to 220 mmol) of 1-MCP is needed to treat warehouses of 10 m³ up to 5000 m³. Depending upon the size of the warehouse more than one 1-MCP generating device can be used.

In the 1-MCP generating device, the amount of 1-MCP precursor is in the range of about 50 mg to about 30 g, and the amount of fluoride ion-containing compound solution is in the range of about 0.1 ml to about 200 ml, and thus a vessel having a volume ranging from 1 ml to 500 ml may be used as a first vessel and a second vessel.

The flow rate used in the 1-MCP generating device of the present invention has been optimized to obtain both a high yield of 1-MCP and the lowest possible amount of impurities. It has been found that a flow rate of 50+/−10 SCCM is optimal (corresponds to 53.6+/−10.7 cm³/min at a standard temperature and pressure). This optimal flow rate can be used for a 1-MCP generating device of the present invention wherein the first, second and third vessel vary in size from 20 ml to 500 ml. It will be appreciated by the skilled person that for extremely large vessel sizes, the flow rate may need to be adjusted.

Materials and types of the first vessel and the second vessel of the 1-MCP generating device are not particularly limited as long as they have a structure capable of stably storing used materials and, if necessary, discharging the produced materials. For example, the first vessel and the second vessel may be any vessel that includes an inlet and an outlet and is made of an inert material with respect to a material to be stored. In particular, the most widely used resins such as polyethylene and polypropylene may be used in terms of durability, light weight, and economical costs, and a fluorinated resin such as polytetrafluoroethylene (PTFE) may be also used in terms of durability, light weight, handling convenience, and reliability.

### EXAMPLES

### 1) Synthesis of 1-methylcyclopropene (See FIG. 3 for System Set-Up)

First, 150 ml plastic vessels made of polyethylene were prepared for use as a first vessel, a second vessel, and a third vessel, respectively. The plastic vessels were coupled with a cap unit of a 1-MCP generating device such that except for their inlets and outlets, they were sealed. Tubes were inserted into the inlets and outlets of the first vessel, the second vessel, and the third vessel such that the outlet of the first vessel was connected to the inlet of the second vessel, and the outlet of the second vessel was connected to the inlet of the third vessel.

A solution of 18.9 g (=72.3 mmol) of tetrabutylammonium fluoride (TBAF) in 21.5 ml of DMSO was introduced into the first vessel. Trans-1-methyl-1-(methane-sulfonyloxy)-2-(butyldimethylsilyl)cyclopropane (6.5 g=24.6 mmol) as a 1-MCP precursor was injected into the second vessel and the temperature of the second vessel was maintained at 40° C. by using a thermostat. The third vessel was filed with 115 g of a 0.1 M NaOH aqueous solution.

An air pump (Thomas diaphragm air pump model 2002VD/0,5/E/LC) was connected by tubing to the inlet of the first vessel and air was constantly flowed to the first vessel at a flow rate of approximately 250 ml/min for 2 hours. The flow rate was measured by a mass flow sensor (AWM3100 from Honeywell) and a feedback control circuit regulated the speed of the air pump to maintain a constant flow rate. A flow restrictor was an orifice disc with a hole of 0.15 mm diameter.

The gas was discharged via the third vessel from the second vessel and was collected in a 1 m³ container (IBC container). After 2.5 hours a sample was taken from the IBC container and analyzed using the GC procedure below.

The yield of 1-MCP, total amount of impurities and amount of butyldimethylsilylflouoride (main impurity) are listed in the Table 1.

### 2) Synthesis of 1-methylcyclopropene (See FIG. 3 for System Set-Up)

First, 150 ml plastic vessels made of polyethylene were prepared for use as a first vessel, a second vessel, and a third vessel, respectively. The plastic vessels were coupled with a cap unit of a 1-MCP generating device such that except for their inlets and outlets, they were sealed. Tubes were inserted into the inlets and outlets of the first vessel, the second vessel, and the third vessel such that the outlet of the first vessel was connected to the inlet of the second vessel, and the outlet of the second vessel was connected to the inlet of the third vessel.

A solution of 18.9 g (=72.3 mmol) of tetrabutylammonium fluoride (TBAF) in 21.5 ml of DMSO was introduced into the first vessel. Trans-1-methyl-1-(methane-sulfonyloxy)-2-(butyldimethylsilyl)cyclopropane (6.5 g=24.6 mmol) as a 1-MCP precursor was injected into the second vessel and the temperature of the second vessel was maintained at 40° C. by using a thermostat. The third vessel was filed with 115 g of a 0.1 M NaOH aqueous solution.

An air pump (Thomas diaphragm air pump model 2002VD/0,5/E/LC) was connected by tubing to the inlet of the first vessel and air was constantly flowed to the first vessel at a flow rate of approximately 52 ml/min for 2 hours. The flow rate was measured by a mass flow sensor (AWM3100 from Honeywell) and a feedback control circuit regulated the speed of the air pump to maintain a constant flow rate. A flow restrictor was an orifice disc with a hole of 0.15 mm diameter.

The gas was discharged via the third vessel from the second vessel and was collected in a 1 m³ container (IBC container). After 2.5 hours a sample was taken from the IBC container and analyzed using the GC procedure below.

The yield of 1-MCP and the amount of butyldimethylsilylflouoride as the main impurity are listed in the Table 1.

GC Procedure:

TABLE 1

| | flowrate: 250 ml/min | flowrate: 52 ml/min |
|---|---|---|
| Yield 1-MCP | 92.2% | 83.9% |
| butyldimethylsilylfluoride | 2.17% relative to | 0.43% relative to |
| | 1-MCP | 1-MCP |

The lower flow rate in Example 2 of 52 ml/min produces 1-MCP with a much lower amount of impurities compared to the higher flow rate of 250 ml/min used in Example 1.

### 3) Flow Rate Variation Between Mechanical Needle Valve and Pump Regulated with an Electronic Flow Control Circuit and Mass Flow Sensor

a) 1-MCP Generating Device with Mechanical Needle Valve

The 1-MCP generating device as used in Example 2 was equipped with a needle valve for mechanical flow control as used in the prior art device of WO-2012/134088. The flow rate was set to 53 ml/min and the 1-MCP generator was operated for one hour and at various moments over the one hour run period, the flow rate was measured at the outlet (12). The flow rate was measured during a period of one minute and the minimum and maximum flow measured were recorded. Different runs on different days produced a different flow rate and the minimum and maximum flow rate measured has been listed in Table 2.

TABLE 2
flow rate measured at outlet (12) for 1-MCP generator
with needle valve

| | | | |
|---|---|---|---|
| Time | minimum flow rate | maximum flow rate | difference |
| (minute) | (ml/min) | (ml/min) | (ml/min) |

Measurement Day 1

| | | | |
|---|---|---|---|
| 1 | 41 | 47 | 6 |
| 13 | 40 | 48 | 8 |
| 21 | 38 | 48 | 10 |
| 34 | 38 | 48 | 10 |
| 58 | 39 | 48 | 9 |
| average | 39 | 48 | 9 |

Measurement Day 2

| | | | |
|---|---|---|---|
| 1 | 58 | 63 | 5 |
| 7 | 48 | 62 | 14 |
| 21 | 47 | 61 | 14 |
| 37 | 43 | 61 | 18 |
| 51 | 43 | 62 | 19 |
| 68 | 47 | 61 | 14 |
| average | 48 | 62 | 14 |

Measurement Day 3

| | | | |
|---|---|---|---|
| 1 | 14 | 26 | 12 |
| 12 | 14 | 27 | 13 |
| 33 | 16 | 26 | 10 |
| 43 | 14 | 28 | 14 |
| average | 15 | 27 | 12 |

Conclusion: a 1-MCP generating device equipped with a mechanical needle for flow control not only demonstrates a large difference in flow rate measured at the outlet (12) but also large differences in the average flow rate between different measurement days without changes to the initial flow rate setting of 53 ml/min between the measurement days.

b) 1-MCP Generating Device with Mass Flow Sensor and Flow Control Circuit

The 1-MCP generating device of Example 2 was used wherein the flow rate was set to 53 ml/min and the 1-MCP generator was operated for two hours and at various moments over the two hour run period, the flow rate was measured at the outlet (12). The flow rate was measured during a period of one minute and the minimum and maximum flow measured were recorded. Different runs on different days produced a different flow rate and the minimum and maximum flow rate measured has been listed in Table 3.

TABLE 3
flow rate measured at outlet (12) for 1-MCP generator with
mass flow sensor

| | | | |
|---|---|---|---|
| Time | minimum flow rate | maximum flow rate | difference |
| (minute) | (ml/min) | (ml/min) | (ml/min) |

Measurement Day 1

| | | | |
|---|---|---|---|
| 20 | 50 | 57 | 7 |
| 30 | 51 | 56 | 5 |
| 40 | 50 | 57 | 7 |
| 50 | 51 | 56 | 5 |
| 60 | 50 | 56 | 6 |
| 70 | 51 | 56 | 5 |
| 80 | 52 | 56 | 4 |
| 90 | 51 | 56 | 5 |
| 100 | 51 | 57 | 6 |
| 110 | 51 | 56 | 5 |
| 118 | 51 | 57 | 6 |
| average | 51 | 56 | 5.4 |

Measurement Day 2

| | | | |
|---|---|---|---|
| 10 | 53 | 58 | 5 |
| 20 | 53 | 59 | 6 |
| 30 | 53 | 59 | 6 |
| 40 | 53 | 59 | 6 |
| 50 | 53 | 59 | 6 |
| 60 | 52 | 59 | 7 |
| 70 | 54 | 59 | 5 |
| 80 | 55 | 59 | 4 |
| 90 | 53 | 59 | 6 |
| 100 | 53 | 59 | 6 |
| 118 | 54 | 58 | 4 |
| average | 53 | 59 | 5.2 |

Conclusion: a 1-MCP generating device according to the present invention demonstrates a low variation in flow rate at the outlet (12) which differs little from one measurement day to another measurement day. The measured minimum and maximum flow rate was kept within the 20% of the desired flow rate of 53 ml/min.

1. A 1-methylcyclopropene (1-MCP) generating device wherein the flow rate of the carrier gas does not deviate more than 20% from the desired flow rate, comprising:
a first vessel comprising an inlet, an outlet, and a fluoride ion-containing compound of formula (II) 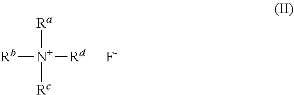
wherein R^{a}, R^{b}, R^{c}, R^{d} are each independently selected from C₁₋₂₀alkyl, phenyl and naphthyl;
a second vessel comprising an inlet, an outlet, and a 1-MCP precursor of formula (I) 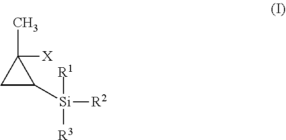
wherein X is halogen, or C₁₋₆alkylS(O)₂O—;
R¹, R², R³ are each independently selected from hydrogen, C₁₋₆alkyl, phenyl, C₁₋₆alkyloxy, and halogen;
a third vessel comprising an inlet, an outlet, and a washing solution;
a pump to supply a carrier gas that is introduced into the first vessel to transfer the fluoride ion-containing compound of formula (II) to the second vessel where said fluoride ion-containing compound of formula (II) reacts with the 1-MCP precursor of formula (I) and the resulting 1-MCP is transferred with the carrier gas to the third vessel where it bubbles through the washing solution before the carrier gas with the 1-MCP is released to the outside;
characterized in that the flow rate of the carrier gas is regulated by a mass flow sensor that provides a flow rate signal to a flow control circuit that provides the pump with a modulation signal to regulate the flow rate of the carrier gas and that a flow restrictor is present between the pump and the mass flow sensor.

2. The device as claimed in claim 1 wherein the flow restrictor is narrow bore tubing having a diameter that is 1/10 of the diameter of the tubing between the pump and the mass flow sensor.

3. The device as claimed in claim 1 wherein the flow restrictor is an orifice disc with a hole having a diameter ranging from 0.10 mm to 0.25 mm, in particular 0.15 mm.

4. The device as claimed in claim 2 wherein the mass flow sensor is placed between the pump and the first vessel.

5. The device as claimed in claim 1 wherein the 1-MCP precursor of formula (I) is trans-1-methyl-1-(methanesulfonyloxy)-2-(butyldimethyl-silyl)cyclopropane.

6. The device as claimed in claim 5 wherein the fluoride ion-containing compound of formula (II) is tetrabutylammonium fluoride (TBAF).

7. The device as claimed in claim 6 wherein tetrabutylammonium fluoride (TBAF) is dissolved in DMSO.

8. The device as claimed in claim 7 wherein the washing solution is an aqueous solution of NaOH, KOH, Na₂CO₃, NaHCO₃, K₂CO₃, KHCO₃, Na₂SiO₂, K₂SiO₂, sodium methanolate, sodium ethanolate, or sodium isopropanolate.

9. The device as claimed in claim 8 wherein the washing solution is a 0.1 M aqueous sodium hydroxide solution.

10. The device as claimed in claim 9 wherein the flow rate is 50±10 CCM.

11. The device as claimed in claim 1 wherein the second vessel is equipped with a heating element.

12. The device as claimed in claim 11 wherein the heating element keeps the second vessel at a temperature ranging from 30° C. to 50° C., in particular 40° C. to 45° C.

13. The device as claimed in claim 1 wherein the first and the second vessel is equipped with a heating element.

14. The device as claimed in claim 12 wherein the heating element keeps the first and second vessel at a temperature ranging from 30° C. to 50° C., in particular 40° C. to 45° C.

15. A method for treating and storing harvested agricultural products using the device as claimed in claim 1.

16. (canceled)

17. A method of inhibiting the action of ethylene, comprising using the device as claimed in claim 1.
